(19) United States
(12) Patent Application Publication
(10) Pub. No.: US 2007/0224634 A1
(43) Pub. Date: Sep. 27, 2007

(54) Methods for Detecting L-Ficolin Dependent Activation of the Lectin Pathway of Complement and Kits Therefor

(75) Inventors: Nicholas J. Lynch, Leicester (GB); Wilhelm J. Schwaeble, Leicester (GB)

Correspondence Address: MORRISON & FOERSTER LLP, 12531 HIGH BLUFF DRIVE, SUITE 100, SAN DIEGO, CA, 92130-2040, US

(73) Assignee: University of Leicester Research And Business Development Office, Leicestershire (GB)

(21) Appl. No.: 10/580,810

(22) PCT Filed: Nov. 25, 2004
(86) PCT No.: PCT/GB04/50030

§ 371 Date: Mar. 16, 2007

(30) Foreign Application Priority Data
Nov. 25, 2003 (GB) 0327368.7

(51) Int. Cl. G01N 33/53; C07C 35/00; C07K 16/00

(52) U.S. Cl. 435/7.1; 530/387.1; 568/700

(57) ABSTRACT

The invention provides methods, assays and kits for detecting L-ficolin dependent activation of the lectin pathway of complement and for identifying L-ficolin abnormalities. Also provided are methods for detection of gram positive bacteria based on the interaction between L-ficolin complexes and LTA.

### METHODS

The invention relates to methods for detecting L-ficolin dependent activation of the lectin pathway of complement, methods for diagnosis of L-ficolin complex deficiencies or functional abnormalities, and methods for detection of gram positive bacteria. Methods of the invention are based on the specific interaction between LTA and L-ficolin complexes, The invention also provides assays comprising methods of the invention, and kits for performing methods and assays of the invention.

### BACKGROUND TO THE INVENTION

The complement system comprises a complex series of potentially interactive blood proteins that are ‘activated’ via two main pathways (1). The ‘classical’ pathway is activated by the pattern recognition molecule, C1q, that binds to certain antigen-antibody complexes. This leads to conformational changes, a cascade of molecular interactions and the generation of key enzymic activity (C3 convertase).

A second, recently described pathway of complement activation, termed the ‘lectin’ pathway, is activated by the interaction of mannose-binding lectin (MBL) and related pattern-recognition molecules with terminal carbohydrate residues on the surface of intact bacteria and other microbes. Unlike the classical pathway, activation of the lectin pathway is not dependent on interaction with antigen-antibody complexes. Lectin pathway activation also involves a cascade of different molecular interactions leading to C3 convertase activity.

A third pathway, the ‘alternative’ pathway, is now recognised as an amplification loop that enhances C3 convertase synthesis following activation of either classical or lectin pathways. C3 convertase acts on C3, an abundant normal blood protein to yield a series of products. Products of C3 conversion include C3b, a fragment that coats microbes (a process termed opsonisation) and enhances their uptake and destruction by phagocytes. Two further products, C3a and C5a (termed anaphylatoxins), are capable of activating mast cells and neutrophils, thereby producing inflammatory reactions. A further product is cytolytic and termed membrane attack complex (MAC or C5b-9). It arises via an additional cascade of events leading to production of hollow tube-like structures that are inserted into the membranes of bacteria and other microbes, thereby generating pores which lead to microbial lysis.

Complement activation pathways are key to defense against infection, individuals with hereditary deficiency of certain complement pathway gene products may be profoundly susceptible to serious infection. The deficiency may manifest as absence of a particular protein or expression of a protein with a reduced or abolished biological function.

The lectin pathway of complement activation provides an essential route of innate anti-microbial host defense. Activation of the lectin pathway occurs in response to carbohydrate structures present on microbial surfaces and is initiated through multi-molecular fluid-phase complexes composed of a carbohydrate recognition subcomponent and the lectin pathway serine protease, MASP-2 (for Mannan binding lectin associated serine protease-2). Three different carbohydrate recognition subcomponents that form complexes and activate complement via MASP-2 have been described: Mannan binding lectin (MBL), L-ficolin, and H-ficolin (previously described as Hakata antigen) (1-4). All recognition subcomponents consist of homotrimers of a single polypeptide chain with an N-terminal collagen-like domain, a neck region, and a C-terminal carbohydrate-binding domain (5). In MBL, this carbohydrate recognition domain (CRD) is a classical C-type lectin domain, while the CRDs of ficolins show a fibrinogen-like domain structure. In plasma, the recognition subcomponents are present as higher-order oligomers of the homotrimeric subunits that form complexes with MASP-2 and two other serine proteases, named MASP-1 and MASP-3, to compose a lectin pathway activation complex (6-9). Of these, only MASP-2 is known to translate the binding of lectin pathway complexes to microbial carbohydrates into activation of complement by cleavage of C4 and C4b bound C2 (8, 10-12). It has been shown that MBL binds to a range of clinically important micro-organisms including fungi, viruses, and both Gram-negative and Gram-positive bacteria (13, 14). In contrast, little is known about the binding specificities of the ficolins. H-ficolin has been shown to bind to S. typhimurium, S. minnesota, and E. coli (15), while L-ficolin has been shown to activate the lectin pathway after binding to S. typhimurium (16). Thus the importance of MBL in anti-microbial host defense is well recognized, but the role of the ficolins remains largely undefined.

Lipoteichoic acid (LTA) is a cell wall constituent found in all gram-positive bacteria. LTA is increasingly regarded as the Gram-positive equivalent of LPS. It is a potent immunostimulant that induces cytokine release from mononuclear phagocytes and whole blood (17, 18). Previous studies have shown that on LTA preparations isolated from various bacterial strains complement activation occurs in an antibody-independent fashion (25-27). The mode of activation, however, remained unclear. Based on measurements demonstrating a significant consumption of complement components C4 and C2 (and a moderate consumption of haemolytically active C1), the most recent of these reports suggested that LTA might activate complement through activation of the classical pathway involving a direct interaction of LTA with C1q (27). No direct binding between LTA and C1q, however, was described.

### STATEMENT OF INVENTION

The present invention provides a method for detecting L-ficolin dependent activation of the lectin pathway of complement comprising:
(a) contacting L-ficolin lectin pathway activation complex with Lipoteichoic acid (LTA) in conditions that permit specific binding thereof and
(b) detecting complement activation.

This method is based on the novel specific interaction between LTA and L-ficolin lectin pathway activation complex.

The present inventors have found that complexes of the lectin pathway recognition subcomponent L-ficolin specifically bind the lipoteichoic acid (LTA) cell wall constituent found in all gram-positive bacteria. L-ficolin complexes can bind to LTA with or without association of MASPs with L-ficolin. In plasma, L-ficolin complexes are normally present as oligomers of L-ficolin trimers and may associate with MBL-associated serine proteases 1, 2, and 3 (i.e. MASP1, MASP2, MASP3) and a small MBL associated protein of 19 kDa (MAp19 or SMAP) to form L-ficolin lectin pathway activation complex (L-ficolin activation complex).

Complement activation via the lectin pathway is mediated through MASP-2, thus complement activation occurs only when the L-ficolin complex is associated with the MASPs and MBL to form L-ficolin lectin pathway activation complex (L-ficolin/MASP). L-ficolin complexes that bind to LTA without being associated to MASPs do not activate the lectin pathway of complement, but are thought to have some biological effects, such as collectin receptor mediated enhancement of phagocytosis.

LTA may be derived from gram positive bacteria, or synthetic LTA may be obtained by chemical synthesis. In preferred methods of the invention, the LTA is immobilised on a support. The L-ficolin activation complex can be obtained from blood, and may be present as a component of whole blood, serum, or an extract therefrom. In blood, L-ficolin lectin pathway activation complex is formed when L-ficolin oligomers associate with MBL-associated serine protease 1 (MASP1), MASP2, MASP3 and a small MBL associated protein.

Conditions that permit specific binding of L-ficolin complex with LTA, in particular specific binding of L-ficolin lectin pathway activation complex with LTA can be achieved using high salt concentrations, e.g. using a high salt buffer (500 mM) to dilute blood serum samples, dissociate C1 and prevent activation of endogenous C4, as described by Petersen et al. (22). Buffers containing metal chelators are a viable alternative, e.g. those described by Hugli et al in U.S. Pat. No. 6,297,024.

In preferred embodiments of the invention, complement activation is detected by a C3 and/or C4 cleavage assay, which may involve detection of a C3 and/or a C4 cleavage product. A suitable method for detection (and quantification) of complement activation is the C4 cleavage assay of Petersen et at (22) as described herein. Alternatively, a method such as that described by Hugli et al in U.S. Pat. No. 6,297,024 (the disclosure of which is incorporated herein by reference) could be employed to detect and quantify complement activation.

The C3 and/or C4 cleavage product can be detected using a ligand specific for the cleavage product, labelled directly or indirectly with a detectable marker Detection systems based on direct or indirect labelling of a ligand specifically bound to a particular target molecule are well known in the art. For direct methods, a ligand specific for the target molecule is itself labelled with a detectable marker. Using indirect detection methods, an unlabelled ligand is incubated with the sample to allow specific binding of the ligand to its target (where present). A second ligand, specific for the first ligand and which is or can be labelled with a detectable marker is provided and allowed to bind to the first ligand. In both direct and indirect detection systems the final stage is detection of the detectable marker.

In preferred embodiments of the method for detecting L-ficolin dependent activation of the lectin pathway of complement invention, the ligand specific for the cleavage product is an antibody or a binding fragment of an antibody (such as an Fab or F(ab′)₂ fragment), more preferably antibody or a binding fragment thereof, specific for a C3 or C4 cleavage product, Complement activation can be detected by detection of the C3 cleavage product C3b, in which instance the ligand is preferably an ant-C3b antibody or a binding fragment of an anti-C3b antibody (i.e. an antibody fragment such as an Fab or F(ab′)2 fragment that specifically binds C3b). Alternatively, or additionally, complement activation can be detected by detection of the C4 cleavage product C4b and/or the C4 cleavage product C4c; the ligand is preferably an anti-C4b antibody, an anti-C4c antibody or a binding fragment thereof (i.e. an antibody fragment such as a Fab or F(ab′)2 fragment that specifically binds C4b or C4c).

Various detectable markers suitable for use in methods of the invention are known in the art; fluorescent, luminescent or radioactive markers are particularly appropriate. The detectable marker can be selected from the group comprising alkaline phosphatase, horse radish peroxidase, biotin, europium, a fluorochrome (such as fluorescein isothiocyanate or a fluorescent protein) or a radiolabel. In a preferred embodiment of the method for detecting L-ficolin dependent activation of the lectin pathway of complement, the detectable marker is alkaline phosphatase and the alkaline phosphatase is detected using a colorimetric substrate, preferably p-nitrophenyl phosphate (pNPP). In an alternative preferred method of the invention, the detectable marker is fluorescein isothiocyanate, which can be detected by fluorescence microscopy.

The invention further provides a functional assay for L-ficolin dependent activation of the lectin pathway of complement, comprising a method as described herein for detecting L-ficolin dependent activation of the lectin pathway of complement. Also provided is a method for identifying the presence of an L-ficolin abnormality in an individual (e.g. a deficiency or absence of L-ficolin or the presence of an aberrant L-ficolin which is unable to form a functional L-ficolin lectin pathway activation complex) comprising a method or assay according to the invention.

Accordingly the invention provides a method for identifying the presence of an L-ficolin abnormality comprising:
(a) contacting LTA with a solution comprising blood, serum or an extract therefrom, in conditions that permit specific binding of L-ficolin lectin pathway activation complex to LTA, and,
(b) detecting and quantifying specific binding of the L-ficolin lectin pathway activation complex to LTA.

Methods, assays and kits of the invention may include suitable positive and negative controls. Methods and assays may include comparison of the amount of specific L-ficolin complex-LTA binding detected in a test solution with the amount of specific L-ficolin complex-LTA binding found in one or more reference samples, to enable identification of L-ficolin abnormalities, in particular abnormalities that affect binding of L-ficolin complex to LTA.

Using functional methods and assays of the invention, L-ficolin abnormalities that result in failure to activate the lectin pathway of complement activation can be detected. In such functional assays, the presence or absence of specific binding of L-ficolin lectin pathway activation complex to LTA can be detected, and, if desired quantified, by assaying complement activation. The level of complement activation detected in a test solution can be compared with the level of complement activation found in one or more reference samples, to enable identification of L-ficolin abnormalities, in particular abnormalities that reduce or abolish complement activation.

In methods for identifying L-ficolin abnormalities, LTA is preferably immobilised on a support.

A preferred method for detecting and/or quantifying L-ficolin dependent activation of the lectin pathway of complement comprises:

(a) providing LTA immobilised on a solid support, preferably one or more wells on a multiwell plate

(b) contacting the immobilised LTA with a solution comprising blood, serum or an extract therefrom in conditions that permit binding of L-ficolin lectin activation complex to LTA, but prevent activation of endogenous C4, and cause dissociation of the C1 complex.

(c) performing a C3 and/or C4 cleavage assay to detect (and, if desired, to quantify) complement activation.

The invention further provides a method for detecting and/or identifying gram positive bacteria comprising:

(a) contacting a sample comprising bacteria or suspected of comprising bacteria with L-ficolin complex in conditions that permit specific binding of L-ficolin complex to LTA, and,

(b) detecting specific binding of L-ficolin complex to LTA present on gram positive bacteria.

In this embodiment the L-ficolin complex can be L-ficolin lectin pathway activation complex, or a non-activating L-ficolin complex i.e. an oligomer of L-ficolin trimers without associated MASPs.

Specific binding of L-ficolin complex to LTA can be detected using a ligand labelled directly or indirectly with a detectable marker. The ligand is preferably an antibody or a binding fragment of an antibody, more preferably an antibody specific for L-ficolin or a binding fragment thereof. By binding fragment is meant a fragment of an antibody that retains the ability to specifically bind a target molecule normally bound by the full antibody. A particularly preferred ligand for detection of specific binding of L-ficolin complex to LTA on gram positive bacteria is an antibody specific for human L-ficolin, such as GN4 or GN5 (commercially available through Hycult biotechnology b.v., The Netherlands), or a fragment thereof that specifically binds L-ficolin. The detectable marker can be, for example, a fluorescent, luminescent or radioactive marker, and is preferably selected from the group comprising alkaline phosphatase, horse radish peroxidase, biotin, europium, a fluorochrome (such as fluorescein isothiocyanate or a fluorescent protein) or a radiolabel. In a preferred embodiment, the detectable marker is alkaline phosphatase and the alkaline phosphatase is detected using a calorimetric substrate, preferably p-nitrophenyl phosphate (pNPP). An alternative preferred detectable marker is a fluorochrome (such as fluorescein isothiocyanate) which can be detected by fluorescence microscopy.

Immobilized LTA from Staphylococcus aureus binds L-ficolin complexes from sera, and these complexes initiate lectin pathway-dependent C4 turnover. C4 activation correlates with serum L-ficolin concentration, but not with serum MBL levels, L-ficolin binding and corresponding levels of C4 turnover were observed on LTA purified from other clinically important bacteria, including Streptococcus pyogenes and Streptococcus agalactiae. None of the LTA preparations bound MBL, H-ficolin or the classical pathway recognition molecule C1 q. This demonstrates that the L-ficolin/LTA interaction initiates an innate anti-microbial immune response by triggering the lectin pathway of complement activation.

Methods and assays according to the invention can be performed in multiwell format, 96 well format is preferred. Methods or assays according to the invention can be performed in high throughput format.

Also provided are kits for performing methods and assays according to the invention. One such kit is provided for detecting L-ficolin dependent activation of the lectin pathway comprising:
(a) LTA immobilised on a support,
(b) a purified C4 or crude C4/C3 preparation, and
(c) a reagent or reagents for detection of a C3 and/or C4 cleavage product, and
(d) optionally, standard serum or purified L-ficolin/MASP complex suitable for generation of a standard curve, and,
(e) optionally instructions for use of the kit.

The support on which LTA is immobilised may comprise one or more wells on a multiwell plate. A reagent for detection of a C3 and/or C4 cleavage product may comprise a ligand capable of being labelled directly or, indirectly with a detectable marker. The ligand can be an antibody or a binding fragment of an antibody. In one form of the kit the ligand specifically binds the C3 cleavage product C3b. Alternatively or additionally, the kit may contain a reagent comprising a ligand specifically binds the C4 cleavage product C4b or the C4 cleavage product C4c.

A kit is provided according to the invention for detecting or for quantifying L-ficolin comprising:
(a) LTA immobilised on a support, and
(b) a reagent or reagents for detection of L-ficolin complex-LTA binding, and
(c) optionally standard serum or purified L-ficolin/MASP complex or purified L-ficolin suitable for generation of a standard curve, and
(d) optionally instructions for use of the kit.

In this embodiment the L-ficolin complex can be L-ficolin lectin pathway activation complex, or a non-activating L-ficolin complex i.e. an oligomer of L-ficolin trimers without associated MASPs.

A reagent for detection of L-ficolin complex-LTA binding may comprise a ligand capable of being labelled directly or indirectly with a detectable marker, suitably the ligand is an antibody or a binding fragment of an antibody, most suitably an antibody or a binding fragment thereof specific for L-ficolin, in particular specific for human L-ficolin for example the antibody GN4 or GN5 (Hycult biotechnology b.v.) or a fragment thereof that specifically binds L-ficolin. Detection of L-ficolin-LTA binding may be performed using ligand labelled directly with a detectable marker, or using ligand labelled indirectly with a detectable marker. A suitable detectable marker would be a fluorescent, luminescent or radioactive marker which can be selected from the group comprising alkaline phosphatase, horse radish peroxidase, biotin, europium (e.g. for time resolved immunofluorometric assays, TRIFMA), a fluorochrome (such as fluorescein isothiocyanate or a fluorescent protein) or a radiolabel. In a preferred embodiment the detectable marker is alkaline phosphatase and the alkaline phosphatase is detected using a calorimetric substrate, preferably p-nitrophenyl phosphate (pNPP). In an alternative preferred embodiment, the detectable marker is fluorescein isothiocyanate, which can be detected by fluorescence microscopy.

Methods assays and kits of the invention can be used to detect and diagnose inherited or acquired immunodeficiency caused by functional deficiencies of serum L-ficolin. These functional deficiencies result in an increased susceptibility to infectious disease.

### DESCRIPTION OF THE FIGURES

FIG. 1 shows that LTA purified from S. aureus binds L-ficolin and activates the lectin pathway. Plates were coated with 1 μg/well LTA or 1 μg/well mannan in carbonate buffer. Diluted sera were added and C4b deposition was measured as described in the C4 cleavage assay. Panel A: C4 activation on LTA with pooled normal human serum (NHS), pooled MBL-deficient serum (MBL−/−) and C1q-depleted pooled NHS (results representative of three independent experiments). Panel B: Comparison of C4 activation on LTA by 12 normal and 6 MBL-deficient (≦50 ng/ml MBL) sera. Panel C: Correlation between C4 activation and serum L-ficolin concentration for the same 18 sera. Results shown are means of duplicates and are relative to the standard serum. Panel D: Inhibition of C4 activation on LTA by pre-incubation of serum with excess fluid-phase LTA or mannan. Results are the means of two independent experiments using normal serum. (Relative C4 activation=1 for uninhibited serum).

FIG. 2 shows that neither H-ficolin nor C1q bind to LTA from S. aureus. Panel A: Microtiter plates were coated with the Hakata Ag specific MAb 4H5 (1 μg/well), PSA from Aerococcus viridans (2 μg/well), LTA from S. aureus (1 μg/well) or formalin-fixed S. aureus (100 μl/well at OD₅₅₀=0.5). Normal serum was diluted in a buffer with physiological salt concentration and added to the plate. H-ficolin binding was assayed by ELISA using polyclonal anti-H-ficolin IgG. Results are the means of two independent experiments and are normalized to 4H5. Panel B: Plates were coated with BSA/anti-BSA immune complexes (IC) or LTA from S. aureus. Normal or C1q-depleted serum was added and C1q binding determined by ELISA.

FIG. 3 shows that purified L-ficolin/MASP complexes bind to LTA and activate C4. Microtiter plates were coated with the L-ficolin specific mAb GN4 (1 μg/well), LTA from S. aureus (1 μg/well), mannan (1 μg/well), PSA from Aerococcus viridens (2 μg/well), or formalin-fixed S. aureus (100 μl/well at OD₅₅₀=0.5). Increasing concentrations of L-ficolin/MASP complexes were added to the wells, and bound L-ficolin (panel A) or C4 activation (panel B) assayed as described in materials and methods. Results are the means of duplicates and are representative of three independent experiments.

FIG. 4 shows that C4 cleavage and L-ficolin binding by LTA from different Gram-positive bacteria. Plates were coated with 1 μg/well of purified LTA from the species and strains indicated. Diluted standard serum was added, and C4 deposition or L-ficolin binding assayed. Results are relative C4 cleavage and relative L-ficolin binding, normalized to LTA from S. aureus (n=4, error bars represent the SD).

FIG. 5 shows that S. aureus binds L-ficolin and activates the lectin pathway of complement. S. aureus DSM20233 was incubated with purified L-ficolin in the presence of various concentrations of purified LTA. L-ficolin binding was detected by flow cytometry using the F(ab)′₂ of MAb 2F5 and FITC-conjugated anti-mouse IgG F(ab)′₂. Panel A: black peak; negative control (no L-ficolin), solid line; L-ficolin (without LTA), dashed line; L-ficolin pre-incubated with 8 mg/ml fluid-phase LTA. Panel B: inhibition of L-ficolin binding to S. aureus by LTA. Results are the means of three independent experiments, error bars represent the SD and the solid line shows binding as a percentage of that seen for L-ficolin alone. Panel C: Inhibition of C4 activation on microtiter plates coated with formalin-fixed S. aureus. Normal serum was pre-incubated with various amounts of LTA, mannan, or LTA and mannan (abscissa) then added to the coated plates and C4 activation assayed as described in the C4 cleavage assay. Results are means of two independent experiments and are relative to the serum with no added inhibitors.

### EXAMPLES

Materials

Unless otherwise stated, all reagents were obtained from Sigma-Aldrich (St. Louis, Mo.). Sera were collected from healthy volunteers, with the approval of the institutional ethical review board, and were assayed for MBL as described by Haurum et al. (19). C1q-depleted serum was prepared from pooled NHS using protein A-coupled Dynabeeds™ (Dynal Biotech, Oslo, Norway) coated with rabbit anti-human C1q IgG (Dako, Glostrup, Denmark), according to the supplier's instructions. L-ficolin was purified from human serum as previously described (16), and its concentration was determined using a proprietary Lowry assay kit (Sigma-Aldrich). PSA, a polysaccharide produced by Aerococcus viridans, was prepared as previously described (20). Formalin-fixed S. aureus DSM20233 were prepared as follows: bacteria were grown overnight at 37° C. in tryptic soy blood medium, washed three times with PBS, then fixed for 1 h at room temperature in PBS/0.5% formalin, and washed a further three times with PBS, before being re-suspended in 15 mM Na₂CO₃, 35 mM NaHCO₃, pH 9.6 (coating buffer).

Extraction and Purification of LTA

Pure LTA, free from endotoxin and other contaminants, was purified from cell extracts of S. aureus (DSM20233), B. subtilis (DSM1087) Bifidobacterium animalis (MB254), S. pyogenes (GAS), and two clinical isolates of S. agalactiae (GBS 6313 and GBS COH1), as previously described (21). The purity of the LTA was greater than 99%, according to nuclear magnetic resonance and mass spectrometry.

C4 Cleavage Assay.

Lectin pathway activation was quantified using the C4 cleavage assay developed by Petersen et al. (22). Briefly, the wells of a Nunc MaxiSorb microtliter plate (Nalge Nunc International, Rochester, N.Y.) were coated with: 100 μl of formalin-fixed S. aureus DSM20233 (OD₅₅₀=0.5) in coating buffer, 1 μg of the H-ficolin specific MAb 4H5 (4) in coating buffer, 1 μg mannan in coating buffer, 1 μg LTA in 100 μl of coating buffer, or 2 μg LTA in 20 μl of methanol. After overnight incubation, wells were blocked with 0.1% HSA in TBS (10 mM Tris-Cl, 140 mM NaCl, pH 7.4), then washed with TBS containing 0.05% Tween 20 and 5 mM CaCl₂ (wash buffer). Serum samples were diluted in 20 mM Tris-Cl, 1M NaCl, 10 mM CaCl₂) 0.05% Triton™-X-100, 0.1% HSA, pH 7.4, which prevents activation of endogenous C4 and dissociates the C1 complex (composed of C1q, C1r and C1s). The diluted samples were added to the plate and incubated overnight at 4° C. The next day, the plates were washed thoroughly with wash buffer, then 0.1 μg of purified human C4 (23) in 100 μl of 4 mM barbital, 145 mM NaCl, 2 mM CaCl₂, 1 mM MgCl₂, pH 7.4 was added to each well. After 1.5 h at 37° C., the plates were washed again, and C4b deposition was detected using alkaline phosphatase-conjugated chicken anti-human C4c (Immunsystem AB, Uppsala, Sweden) and the colorimetric substrate pNPP (p-nitrophenyl phosphate).

Solid-Phase Binding Assays.

Nunc Maxisorb microtiter plates were coated with: LTA, MAb 4H5, or formalin-fixed S. aureus as described above, PSA from Aerococcus viridans (2 μg/well in coating buffer), or immune complexes generated in situ by coating with BSA (1 μg/well in coating buffer), then adding rabbit anti-BSA (2 μg/ml in wash buffer). Wells were blocked with 300 μl of 0.1% HSA in TBS for 1.5 h at room temperature, then washed with wash buffer. Serum samples were diluted in 100 μl of 10 mM Tris-Cl, 140 mM NaCl, 2 mM CaCl₂, 0.05% Triton X-100, 0.1% HSA pH 7.4, added to the plates and incubated overnight at 4° C. After washing, bound proteins were detected using rabbit anti-human L-ficolin IgG (24), rabbit anti-human H-ficolin antiserum (18), or goat anti-human C1 q (Atlantic Antibodies, Stillwater, Minn.). Secondary antibodies were alkaline phosphatase-conjugated goat anti-rabbit IgG or rabbit anti-goat IgG, as appropriate, and bound antibody was detected using the colorimetric substrate pNPP. A standard serum was included on each plate to allow cross-plate normalization of the results.

L-Ficolin ELISA

Nunc Maxisorb microtiter plates were coated with 1 μg/well of the L-ficolin specific MAb GN4 (3) in coating buffer. Wells were blocked, diluted serum samples added, and L-ficolin detected using rabbit anti-human L-ficolin IgG (24), as described above.

Flow Cytometry

100 μl of Staphylococcus aureus DSM20233 (freshly isolated; OD₆₀₀=1.4) were suspended in Veronal buffered saline supplemented with 0.1% gelatin, 2 mM CaCl₂ and 0.5 mM MgCl₂ (GVB), and spun down. The pellets were incubated at 37° C. for 30 min with 20 μl of purified L-ficolin (2 μg/ml) in the presence of various concentrations of LTA, and then washed three times with GVB. The washed cells were then incubated on ice for 30 min with 20 μl of F(ab)′₂ (100 μg/ml) of the anti-human L-ficolin MAb 2F5 (16) and stained on ice for 30 min with 20 μl of fluorescein isothiocyanate-conjugated anti-mouse Igs F(ab)′₂ (100 μg/ml; Dako). The cells were washed twice with GVB between each reaction. Reactivities were evaluated by FACSCalibur 4A flow cytometry (Becton Dickinson, Mountain View, Calif.). F(ab)′₂ fragments of the murine anti-hu man L-ficolin antibody MAb 2F5 (IgG1) were generated by pepsin cleavage using a proprietary kit (Pierce Biotechnology, Rockford, Ill.).

Results

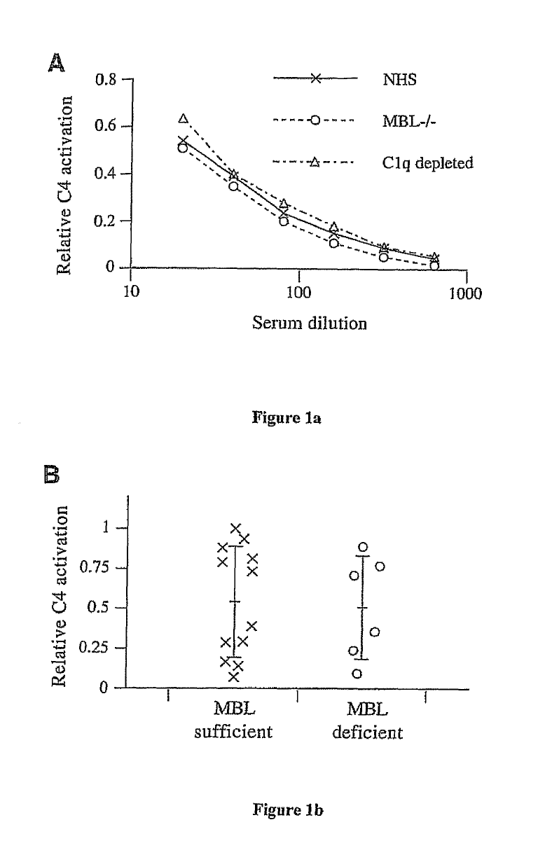

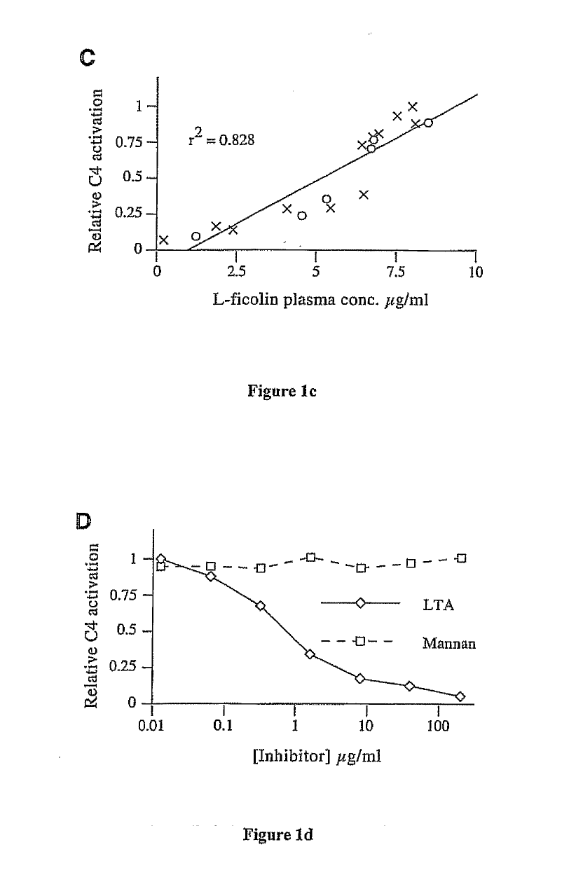

A C4 cleavage assay that monitors complement activation via the lectin pathway was used to determine serum responses to very pure LTA preparations derived from the cell wall of S. aureus strain DSM 20233. As shown in FIGS. 1A and 1B, lectin pathway-mediated C4 cleavage occurred in both MBL-sufficient and MBL-deficient (MBL≦50 ng/ml) sera, suggesting that MBL was not the recognition molecule involved in LTA-dependent complement activation. Similar results were obtained using re-calcified plasma in place of serum (data not shown). Moreover, depletion of C1q had no effect on the ability of serum to activate C4 in response to S. aureus LTA In this assay (FIG. 1A). A sensitive MOL-binding assay detected as little as 50 ng/ml MBL when ELISA wells were coated with mannan, but no MBL binding was detected when wells were coated with LTA from DSM20233 (data not shown). L-ficolin binding to LTA could be demonstrated with all of the sera tested and the level of C4 activation correlated closely with the concentration of L-ficolin in the sera (FIG. 1C). There was no corresponding correlation with the MBL concentrations in these sera. C4 activation on LTA coated wells could be completely inhibited by pre-incubating the serum with excess fluid-phase LTA, while fluid-phase mannan (which inhibits MBL-driven C4 activation) had no effect (FIG. 1D). Initially, the plates were coated with LTA dissolved in methanol, to protect the alkali-labile D-alanine esters on the phosphate backbone, which are essential for LTA-mediated cytokine release (17, 18). However, it was found that L-ficolin binding and C4 activation were similar on LTA that had been dissolved in carbonate buffer at pH 9.2, suggesting that D-alanine substitution is not essential for L-ficolin binding.

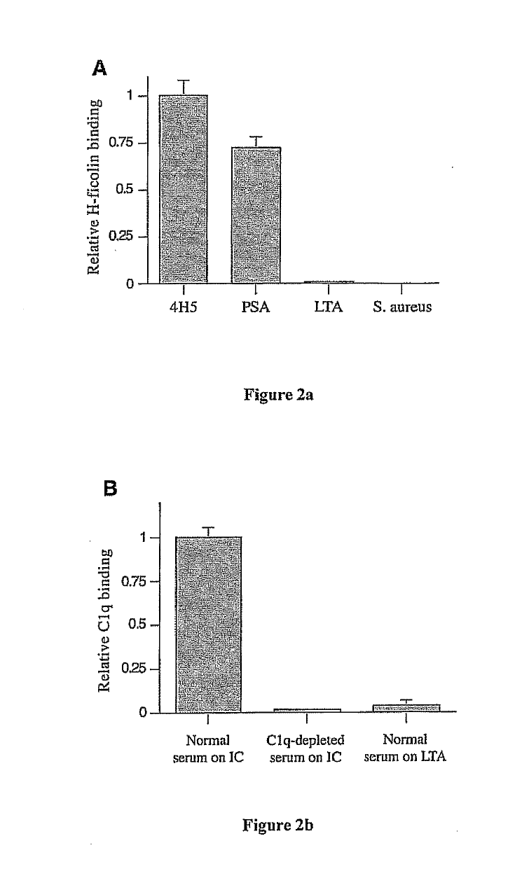

Two experiments demonstrate that H-ficolin does not contribute to the C4 activation seen on LTA from S. aureus strain DSM20233. Firstly, an H-ficolin specific ELISA showed that, although H-ficolin binds to the anti-H-ficolin MAb 4H5 and to PSA from Aerococcus viridans (a known ligand for H-ficolin), it binds neither to whole formalin-fixed DSM20233, nor to LTA from DSM20233 (FIG. 2A). Secondly, coating plates with MAb 4H5 leads to H-ficolin dependent activation of the lectin pathway that can be specifically inhibited by adding excess fluid-phase PSA, but not by adding LTA (data not shown). FIG. 2B illustrates the absence of a direct interaction between C1q and LTA at physiological salt concentrations.

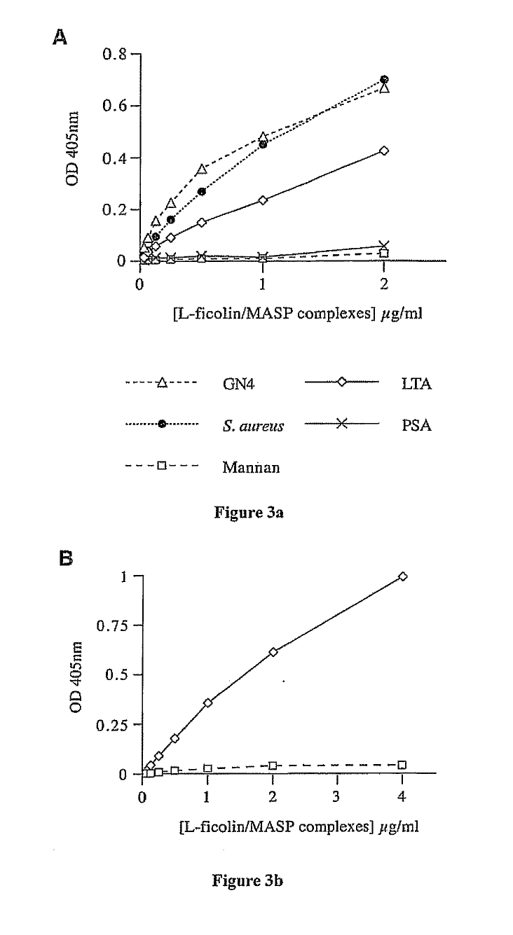

Next, the sera were replaced with purified L-ficolin/MASP complexes (16). Concentration-dependent binding of L-ficolin/MASP complexes was observed on wells coated with the L-ficolin specific mAb GN4, LTA from DSM20233 and formalin-fixed DSM20233, but not on wells coated with PSA or mannan (FIG. 3A). Likewise, concentration-dependent C4 activation was seen on LTA coated wells, but not on those coated with mannan (FIG. 3B).

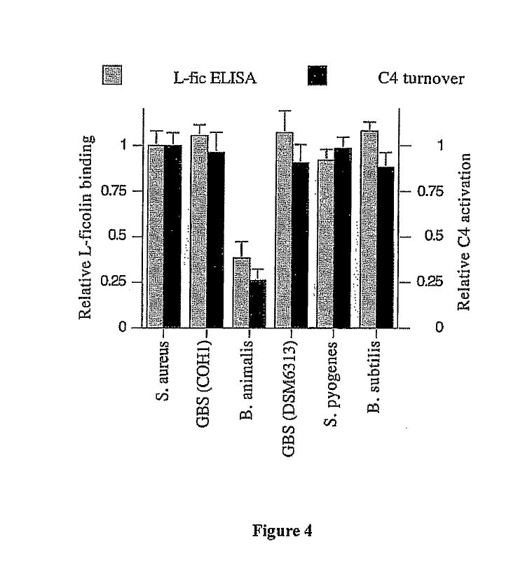

Preparations of pure LTA from other gram positive bacteria were tested for C4 activation, ficolin binding and MBL binding. L-ficolin binding and C4 activation on LTA from B. subtilis (DSM1087), S. pyogenes and S. agalactiae (two isolates) were remarkably similar to that seen for LTA from S. aureus DSM20233 (FIG. 4). LTA from Bifidobacterium animalis bound significantly less L-ficolin, and the C4 activation was correspondingly low. Neither MBL nor H-ficolin bound to any of the LTA preparations tested (data not shown).

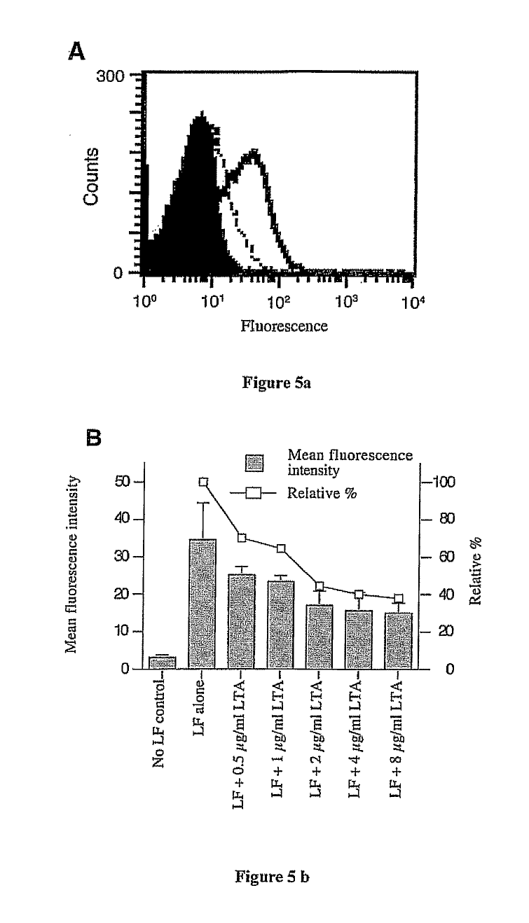

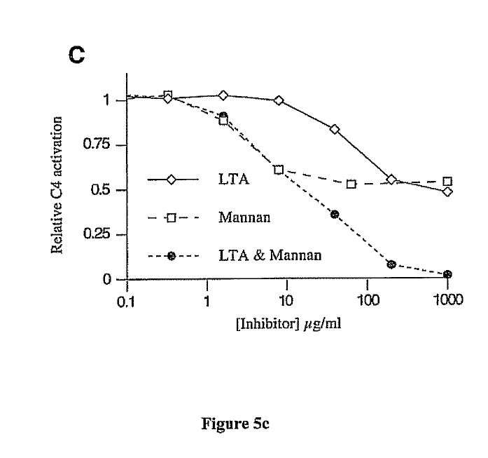

Flow cytometry was used to demonstrate binding of purified L-ficolin/p35 to whole S. aureus DSM20233, and this binding could also be inhibited by excess fluid-phase LTA (FIGS. 5A and 5B). C4 activation on whole formalin-fixed DSM20233 could be inhibited to roughly equal extents by both mannan and LTA (FIG. 50), and the effect of the two inhibitors was additive, implying that approximately half of the C4 activation observed on the whole bacteria is a consequence of MBL binding to cell wall components other than LTA, probably to the mannose-rich peptidoglycan.

Discussion

The experimental work demonstrates that complement activation occurs via the lectin pathway through specific binding of L-ficolin to LTA preparations from different Gram-positive bacterial strains, including S. aureus strain DSM 20233. The binding of L-ficolin to LTA was highly specific, none of the LTA preparations bound MBL or H-ficolin. These findings are consistent with those from Polotsky and co-workers (28), who reported that recombinant human MBL binds to LTA from Enterococcus spp. (in which the polyglycerophospate chain is substituted with glycosyl groups), but not to LTA from nine other species, including S. aureus, S. pyogenes and Bifidobacterium.

Inhibition assays indicated that L-ficolin is responsible for approximately 50% of the total lectin pathway-dependent C4 activation seen on whole formalin-fixed S. aureus; the remaining C4 activation could be inhibited with mannan, and is therefore attributable to MBL binding to cell wall components other than LTA, This finding may explain the observation that the deposition of C4 and iC3b on S. aureus, and the opsonophagocytosis of S. aureus, in MBL-deficient serum is approximately half of that seen in MBL-deficient serum reconstituted with MBL-MASP complexes (29).

The levels of L-ficolin binding and lectin pathway-dependent C4 activation detected on LTA purified from B. subtilis, S. pyogenes, and S. agalactiae were similar to those seen on LTA from S. aureus, while LTA from Bifidobactedum animalis had a reduced capacity to bind serum L-ficolin (approximately 30% of the amount bound by the same concentration of the other LTAs tested), and showed correspondingly reduced C4 activation. The relatively low level of binding to Bifidobacterium LTA is probably a consequence of its backbone structure; Bifidobacterium spp LTA differs from the others in that its backbone consists of lipofuranan instead of polyglycerophospate and it is substituted with monoglycerophospate groups instead of N-acetylated carbohydrate groups (30).

The repertoire of microbial organisms recognized by L-ficolin could both overlap and extend that recognized by MBL. The ability of several fluid-phase carbohydrate recognition molecules to initiate the lectin pathway of complement activation in response to different pathogen-associated molecular patterns broadens the spectrum for the innate response towards invading microbial organisms.

### REFERENCES

1. Ikeda K., T. Sannoh, N. Kawasaki, T, Kawasaki, and I. Yamashina. 1987. Serum lectin with known structure activates complement through the classical pathway. J. Biol. Chem. 262:7451.
2. Matsushita M., and T. Fujita. 1992. Activation of the classical complement pathway by mannose-binding protein in association with a novel C1s-like serine protease. J. Exp. Med, 176:1497.
3. Matsushita M., Y. Endo, and T. Fujita. 2000. Cutting edge: Complement-activating complex of ficolin and mannose-binding lectin-associated serine protease. J. Immunol. 164:2281.
4. Matsushita M., M. Kuraya, N. Hamasaki, M. Tsujimura, H. Shiraki, and T. Fujita. 2002. Activation of the lectin complement pathway by H-ficolin (Hakata antigen). J. Immunol 168:3502.
5. Matsushita M., and T. Fujita. 2002. The role of ficolins in innate immunity. Immunobiology 205:490.
6. Takada F., Y. Takayama, H. Hatsuse, and M. Kawakami. 1993. A new member of the C1s family of complement proteins found in a bactericidal factor, Ra-reactive factor, in human serum. Biochem. Biophys. Res. Commun. 196:1003.
7. Sato T., Y. Endo, M. Matsushita, and T. Fujita. 1994. Molecular characterization of a novel serine protease involved in activation of the complement system by mannose-binding protein. Int. Immunol 6:665.
8. Thiel S., T. Vorup-Jensen, C. M. Stover, W. Schwaeble, S. B. Laursen, K. Poulsen, A. C. Willis, P. Eggleton, S. Hansen, U. Holmskov, K. B. Reid, and J. C. Jensenius. 1997. A second serine protease associated with mannan-binding lectin that activates complement Nature 386:506.
9. Dahl M. R., S, Thiel, M. Matsushita, T. Fujita, A. C. Willis, T. Christensen, T. Vorup-Jensen, and J. C. Jensenius. 2001. MASP-3 and its association with distinct complexes of the mannan-binding lectin complement activation pathway. Immunity 15:127.
10. Vorup-Jensen T., S. V. Petersen, A. G. Hansen, K. Poulsen, W. Schwaeble, R. B. Sim, K. B. Reid, S. J. Davis, S. Thiel, and J. C. Jensenius. 2000. Distinct pathways of mannan-binding lectin (MBL)- and C1-complex autoactivation revealed by reconstitution of MBL with recombinant MBL-associated serine protease-2. J. Immunol. 165:2093.
11. Rossi V., S. Cseh, I. Bally, N. M. Thielens, J. C. Jensenius, and G. J. Arlaud. 2002. Substrate specificities of recombinant mannan-binding lectin-associated serine proteases-1 and -2. J. Biol. Chem, 276:40880.
12. Schwaeble W, J., Dahl, M. R., Thiel, S., Stover, C. M., and Jensenius, J. C. 2002. The Mannan-Binding Lectin-associated Serine Proteases (MASPs) and MAp19: Four Components of the Lectin Pathway Activation Complex encoded by two genes. Immunobiology 205.455.

13. Neth O., D. L. Jack, A. W. Dodds, H. Holzel, N. J. Klein, and M. W. Turner. 2000. Mannose-binding lectin binds to a range of clinically relevant microorganisms and promotes complement deposition. Infect Immun. 68:688.
14. Jack D. L., N. J. Klein and M. W. Turner. 2001. Mannose-binding lectin: targeting the microbial world for complement attack and opsonophagocytosis. Immuno. Rev. 180:86.
15. Sugimoto R., Y. Yae, M. Akaiwa, S. Kitajima, Y. Shibata, H. Sato, J. Hirata, K. Okochi, K. Izuhare, and N. Hamasaki. 1998. Cloning and characterization of the Hakata antigen, a member of the ficolin/opsonin p35 lectin family. J. Biol. Chem. 273:20721.
16. Matsushita M., Y. Endo, S. Taira, Y. Sato, T. Fujita, N. Ichikawa, M. Nakata, and T. Mizuochi. 1996. A novel human serum lectin with collagen- and fibrinogen-like domains that functions as an opsonin. J. Biol. Chem, 271:2448.
17. Morath S., A. Stadelmaier, A. Geyer, R. R. Schmidt and T. Hartung. 2002. Synthetic lipoteichoic acid from Staphylococcus aureus is a potent stimulus of cytokine release. J. Exp. Med. 195:1635.
18. Morath S., A. Geyer, I. Spreitzer, C. Hermann and T. Hartung. 2002. Structural decomposition and heterogeneity of commercial lipoteichoic acid preparation, Infect. Immun. 70:938.
19. Haurum J. S., S. Thiel, H. P. Haagsman, S. B. Laursen, B. Larsen and J. C. Jensenius. 1993. Studies on the carbohydrate-binding characteristics of human pulmonary surfactant-associated protein A and comparison with two other collectins: mannan-binding protein and conglutinin. Biochem J. 293:873.
20. Tsujimura M., C. Ishida, Y. Sagara, T. Miyazaki, K. Murakami, H. Shiraki, K. Okochi, and Y. Maeda. 2001. Detection of serum thermolabile beta-2 macroglycoprotein (Hakata antigen) by enzyme-linked immunosorbent assay using polysaccharide produced by Aerococcus viridans. Clin Diagn. Lab. Immunol. 8&454.
21. Morath S., A. Geyer, and T. Hartung. 2001. Structure-function relationship of cytokine induction by lipoteichoic acid from Staphylococcus aureus. J. Exp. Med. 193:393.
22. Petersen S. V. S. Thiel, L. Jensen, R. Steffensen, and J. C. Jensenius. 2001. An assay for the mannan-binding lectin pathway of complement activation. J. Immunol. Methods. 257:107.
23. Dodds A. W. 1993. Small-scale preparation of complement components C3 and C4. Methods Enzymol. 223:46.
24. Le Y., S. H. Lee, O. L. Kon, and J. Lu. 1998. Human L-ficolin: plasma levels, sugar specificity, and assignment of its lectin activity to the fibrinogen-like (FBG) domain. FEBS Lett. 425:367.
25. Tauber J. W., M. J. Polley, and J. B. Zabriskie. 1976. Nonspecific complement activation by streptococcal structures. II. Properdin-independent initiation of the alternate pathway. J. Exp. Med. 143:1352.
26. Wilkinson B. J., Y. Kim, and P. K. Peterson. 1981. Factors affecting complement activation by Staphylococcus aureus cell walls, their components, and mutants altered in teichoid acid. Infect Immun. 232:216.
27. Loos M., F. Clas, and W. Fischer. 1986. Interaction of purified lipoteichoic acid with the classical complement pathway. Infect Immun. 53:595.
28. Polotsky V. Y., W. Fischer, R A. Ezekowitz, and K. A. Joiner. 1996. Interactions of human mannose-binding protein with lipoteichoic acids. Infect. Immun. 64:380.
29. Neth O., D. L. Jack, M. Johnson, N. J. Klein, and M. W. Turner. 2002. Enhancement of complement activation and opsonophagocytosis by complexes of mannose-binding lectin with mannose-binding lectin-associated serine protease after binding to Staphylococcus aureus. J. Immunol. 169:4430.
30. Fischer, W. 1987. ‘Lipoteichoic acid’ of Bifidobacterium bifidum subspecies pennsylvanicum DSM 20239. A lipoglycan with monoglycerophosphate side chains. Eur J. Biochem, 166.639.

1. A method for detecting L-ficolin dependent activation of the lectin pathway of complement comprising:
(a) contacting L-ficolin lectin pathway activation complex with Lipoteichoic acid (LTA) in conditions that permit specific binding thereof, and
(b) detecting complement activation.

2. The method according to claim 1 wherein LTA is immobilized on a support.

3. The method according to claim 1 wherein L-ficolin complex is obtained from blood.

4. The method according to claim 1 wherein complement activation is detected by a C3 and/or C4 cleavage assay.

5. The method according to claim 4 wherein complement activation is detected by detection of a C3 and/or a C4 cleavage product.

6. The method according to claim 5 wherein the C3 and/or C4 cleavage product is detected using a ligand specific for the cleavage product, labelled directly or indirectly with a detectable marker.

7. The method according to claim 6 wherein the ligand specific for the cleavage product is an antibody or a binding fragment of an antibody.

8. The method according to claim 1 wherein complement activation is detected by detection of the C3 cleavage product C3b.

9. The method according to claim 8 wherein the ligand is an anti-C3b antibody or a binding fragment of an anti-C3b antibody.

10. The method according to claim 6 wherein complement activation is detected by detection of the C4 cleavage product C4b.

11. The method according to claim 10 wherein the ligand is an anti-C4b antibody or a binding fragment of an anti-C4b antibody.

12. The method according to claim 6 wherein complement activation is detected by detection of the C4 cleavage product C4c.

13. The method according to claim 12 wherein the ligand is an anti-C4c antibody or a binding fragment of an anti-C4c antibody.

14. The method according to claim 6 wherein the detectable marker is a fluorescent, luminescent or radioactive marker.

15. The method according to claim 6 wherein the detectable marker is selected from the group comprising alkaline phosphatase, horse radish peroxidase, biotin, europium, fluorescein isothiocyanate, a fluorescent protein or a radiolabel.

16. The method according to claim 6 wherein the detectable marker is alkaline phosphatase and the alkaline phosphatase is detected using a calorimetric substrate.

17. The method according to claim 6 wherein the detectable marker is fluorescein isothiocyanate and is detected using fluorescence microscopy.

18-19. (canceled)

20. A method for identifying an L-ficolin abnormality comprising:
(a) contacting LTA with a solution comprising blood, serum or an extract therefrom, in conditions that permit specific binding of L-ficolin lectin pathway activation complex to LTA, and,
(b) detecting, specific binding of the L-ficolin complex to LTA.

21. The method according to claim 20 wherein LTA is on a support.

22. A method for detecting gram positive bacteria comprising:
(a) contacting a sample comprising bacteria or suspected of comprising bacteria with L-ficolin complex in conditions that permit specific binding of L-ficolin to LTA; and
(b) detecting specific binding of L-ficolin complex to LTA present on gram positive bacteria.

23. The method according to claim 22 wherein specific binding of L-ficolin complex to LTA is detected using a ligand labelled directly or indirectly with a detectable marker.

24. The method according to claim 23 wherein the ligand is an antibody or a binding fragment of an antibody.

25. The method according to claim 24 wherein the antibody is an antibody specific for L-ficolin or a binding fragment of an antibody specific for L-ficolin.

26. The method according to claim 25 wherein the antibody is GN4 or GN5 or a fragment thereof that specifically binds L-ficolin.

27. The method according to claim 23 wherein the detectable marker is a fluorescent, luminescent or radioactive marker.

28. The method according to claim 23 wherein the detectable marker is selected from the group comprising alkaline phosphatase, horse radish peroxidase, biotin, europium, fluorescein isothiocyanate, a fluorescent protein and a radiolabel.

29. The method according to claim 28 wherein the detectable marker is alkaline phosphatase and the alkaline phosphatase is detected using a colorimetric substrate.

30. The method according to claim 28 wherein the detectable marker is fluorescein isothiocyanate and is detected using fluorescence microscopy.

31. The method of claim 1, wherein the method is performed in a 96 well format.

32. The method of claim 1, wherein the method is performed in a high throughput format.

33. (canceled)

34. The kit detecting L-ficolin dependent activation of the lectin pathway comprising:
(a) LTA on a support;
(b) a purified C4 or crude C4/C3 preparation;
(c) a reagent or reagents for detection of a C3 and/or C4 cleavage product;
(d) standard serum or purified L-ficolin/MASP complex suitable for generation of a standard curve; and
(e) instructions for use of the kit.

35. The kit according to claim 34 wherein a reagent for detection of a C3 and/or C4 cleavage product comprises a ligand capable of being labelled directly or indirectly with a detectable marker.

36. The kit according to claim 35 wherein the ligand is an antibody or a binding fragment of an antibody.

37. The kit according to claim 35 wherein the ligand specifically binds the C3 cleavage product C3b.

38. The kit according to claim 35 wherein the ligand specifically binds the C4 cleavage product C4b.

39. The kit according to claim 35 wherein the ligand specifically binds the C4 cleavage product C4c.

40. The kit according to claim 33 for detecting L-ficolin complex comprising:
(a) LTA immobilised immobilized on a support;
(b) a reagent or reagents for detection of L-ficolin complex-LTA binding; and
(c) standard serum or purified L-ficolin/MASP complex or purified L-ficolin, suitable for generation of a standard curve.

41. The kit according to claim 40 wherein the reagent for detection of L-ficolin-LTA binding comprises a ligand capable of being labelled directly or indirectly with the detectable marker.

42. The kit according to claim 41 wherein the ligand is an antibody or a binding fragment of an antibody.

43. The kit according to claim 42 wherein the antibody or binding fragment of an antibody is specific for L-ficolin.

44. The kit according to claim 43 wherein the antibody is GN4 or GN5 or is a binding fragment thereof that specifically binds L-ficolin.

45. The kit according to claim 35 wherein the ligand is labelled directly with the detectable marker.

46. (canceled)

47. The kit according to claim 35 wherein the detectable marker is a fluorescent, luminescent or radioactive marker.

48. The kit according to claim 35 wherein the detectable marker is selected from the group consisting of alkaline phosphatase, horse radish peroxidase, biotin, europium, fluorescein isothiocyanate, a fluorescent protein and a radiolabel.

49. The kit according to claim 35 wherein the detectable marker is alkaline phosphatase and a calorimetric substrate for detection of alkaline phosphatase is provided.

50. The kit according to claim 35 wherein the detectable marker is fluorescein isothiocyanate.

51. The kit according to any of claim 33 wherein the support is one or more wells on a multiwell plate.
